(19) United States
(12) Patent Application Publication
(10) Pub. No.: US 2008/0099576 A1
(43) Pub. Date: May 1, 2008

(54) Decorative air freshener designed to resemble a compact disc

(76) Inventor: Lance G. Hart, Southhaven, MS (US)

Correspondence Address: CHRISTOPHER D. HARRINGTON, SUITE 350, 5300 CORPORATE GROVE DR SE, GRAND RAPIDS, MI, 49512, US

(21) Appl. No.: 11/582,584

(22) Filed: Oct. 19, 2006

(51) Int. Cl. A61L 9/04; A24F 25/00

(52) U.S. Cl. 239/53; 239/54; 239/34

(57) ABSTRACT

A novel car air freshener that simulates a CD-like product, comprises a disc body, a fragrance impregnated disc and a micro perforated disc, all of which are sandwiched to integrally form a car air freshener that emits a scented fragrance. The side of the micro perforated disc that is exposed to the exterior is covered with a plastic film during shipment and storage in order to promote the shelf life of the product. An attached elastic strap is installed on the product to allow the car air freshener to be suspended from a fixture within a vehicle.

### RELATED APPLICATIONS

Not applicable.

### STATEMENT REGARDING GOVERNMENTALLY FUNDED WORK

Not applicable.

### BACKGROUND OF THE INVENTION

The present invention relates to air fresheners for motor vehicles, recreational vehicles, boats, and other confined spaces. More specifically, the present invention relates to an air freshener that has the appearance of a compact disc (“CD”).

Air fresheners for motor vehicles have enjoyed a history that dates back decades, probably to the time when the typically car became an enclosed vehicle. The need to counteract fumes, musty smells of weathered fabrics, concentrated tobacco smoke, and other odor was soon evident and methods for masking or eliminating these odors from the cabin of the automobile were developed early on. A longtime favorite has been the “pine tree” type of car deodorant, which is actually known as the, “Little Tree Car Air Freshener”® and has been manufactured by the Car-Freshener® Corporation since 1952. The Little Tree product is basically an absorbent pad in the shape of a pine tree and has been impregnated with a pleasing fragrance. The product is offered in a sealed packaged in order to preserve its potency and once opened and-placed-into use, the fragrance is allowed to evolve, from the pad and into the cabin of the car.

Various other types of scent dispensers are known in the prior art; some of which are used as car air fresheners and are typically fashioned into whimsical shapes or caricatures. These are somewhat like the Little Tree air freshener in that they rely on an absorbent pad to emit the fragrance over a period of time. Other types of air fresheners have been located in the prior art that comprise devices that are used in the home. Some of these use a semi-solid “wick” that dispenses a scent and the amount of scent being evolved; can be controlled by exposing more of the wick, as per the desires and tastes of the user.

There are also devices that are capable of carrying a scent, whether this is for the purposes of providing a fragrant scent or for evolving a scent used for hunting, and this products have been formed as discs that have honeycombed areas loaded with the particular materials that are used to promote the intended result. While such items bear a rough physical similarity to the present invention, they are really quite different in application and do not possess the degree of utility of the applicant's concept.

There has been a long-standing problem with respect to car air fresheners in providing convenient and easily accessible storage for these products within the vehicle. Many times the prior art products have been relegated to the glove compartment or the center console where they are packed in with other items and end up becoming mangled, or in some cases, having their protective packaging broken, thus eliminating the effectiveness of the freshener when it is time to put it into use. The novel car air freshener of the present invention provides a convenient and effective solution to this problem and- does in a from that is recognizable to most people.

### SUMMARY OF THE INVENTION

A novel car air freshener of the present invention comprises a disc that is cosmetically similar to a CD in appearance, and which includes a lamination of a disc body, a felt disc, and a micro perforated disc, where the felt disc is impregnated with a fragrant scent-that is capable of being emitted-through the micro perforations. The car air freshener of the present invention is sealed with a plastic membrane that hermetically encloses the fragrant scent and maintains the car air freshener in optimal condition until it is deployed for use.

The car air freshener of the present invention is constructed to be the same size and general reconfiguration as a conventional, CD and is packaged in a way to leverage this fact. In one embodiment, the car air freshener is packaged in a jewel case of the type usually containing a commercial CD such as one used for music. The car air freshener can then easily be stored-in a CD holder in the vehicle and retrieved for use by the owner at his/her leisure.

These and-other attributes and-features of the present-invention will-be more fully disclosed herein.

### BRIEF DESCRIPTION OF THE DRAWINGS

FIG. 1 is a front elevational view of the car air freshener of the present invention, shown with an elastic strap for attachment purposes.

FIG. 2 is an exploded isometric view of the car air freshener of the present invention, displaying the components of the invention.

FIG. 3 is a partial-cross sectional view of the fragrance impregnated disc of the car air freshener of the present invention.

FIG. 4 is a partial cross sectional-view of the micro perforated disc of the car air freshener of the present invention.

### DETAILED DESCRIPTION OF THE PREFERRED EMBODIMENTS

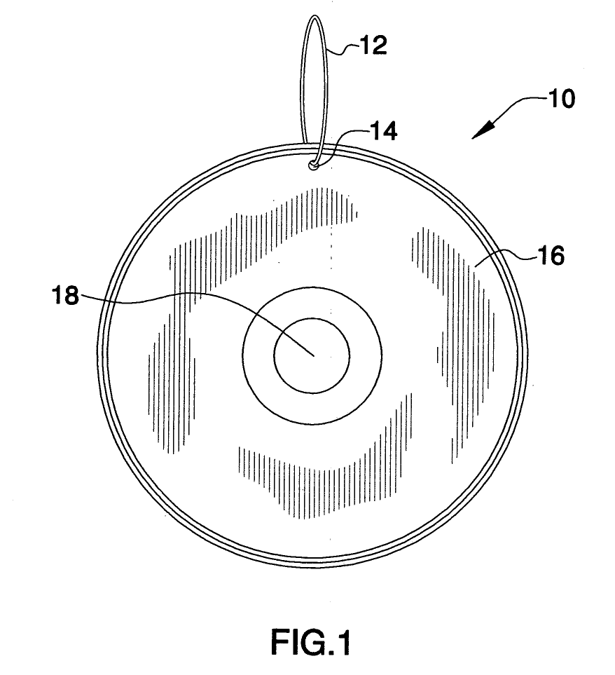

A novel car air freshener 10 in accordance with the present invention is shown generally in FIG. 1, with the elastic strap 12, the mounting hole 14, the disc body 16 and the center hole 1:

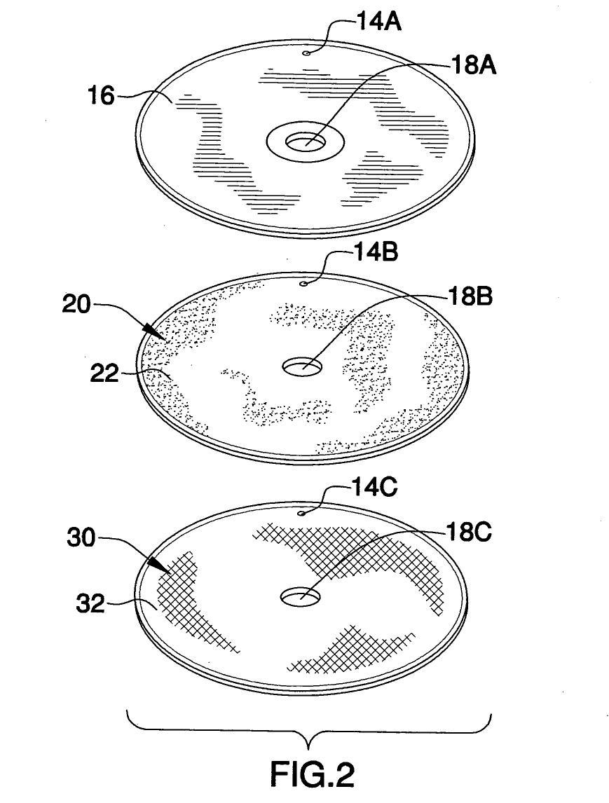

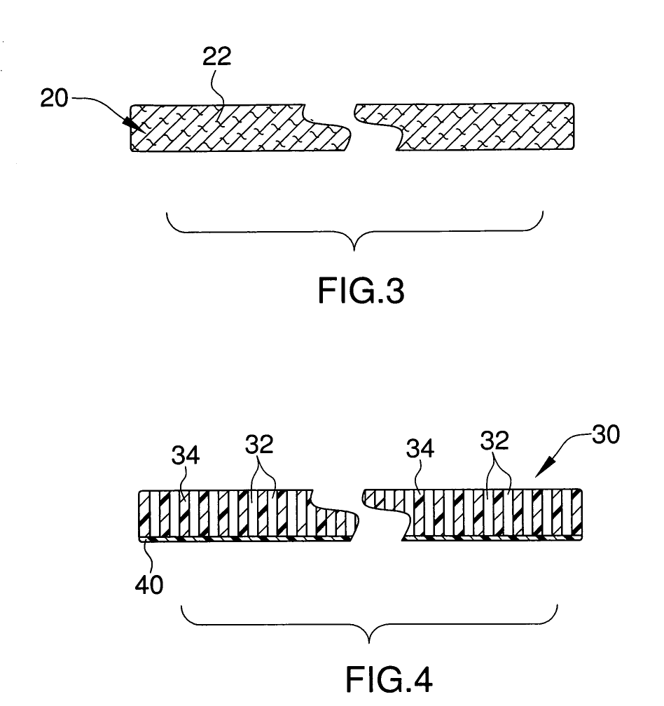

The car air freshener is shown in exploded form in FIG. 2, with the fragrance disc 20; the felt 22. The micro perforated disc 30 is shown with micro perforations 32. The fragrance disc 20 and the micro perforation disc 30 are disclosed in cross section in FIGS. 3 and 4 respectively, and the micro perforations 32 can be seen in detail with adjacent walls 34. The plastic film 4(r is identified as disposed on one side of the micro perforation disc. Features common to each of the disc body 16, the fragrance disc 20 and the micro perforation disc 30 are respectively called out as the mounting holes 14(a), 14(b) and 14(c), and center holes 18(a), 18(b) and 18(c).

The car air freshener of the present invention can be understood as providing scented-fragrance for use in select confined-spaces. While the primary use is directed-towards cars, trucks and other types of motor vehicles, the invention may also be used in boats, campers, closets, bedrooms, and nearly any other suitable place where a scented fragrance would-be desired. For all purposes within this specification, the reference to a “car” or “motor vehicle” with respect to the present invention will be, understood to include all of the anticipated uses and locations.

The car air freshener is comprised of three basic disc components. The disc body is for all outward appearance, a CD type disc, constructed preferably of plastic. The disc body is not perforated and seals one side of the car air freshener. It does provide strength to the assembly and-is part of the lamination that results at the end-of the manufacturing process. The middle disc is the fragrance-impregnated disc which is basically made of a felt body and has is saturated with scent chemistry. This disc, like all of the discs, has the same center hole placement and-is substantially, although not identically, the same diameter. Lastly, the micro perforation disc is the last component of consideration and it includes numerous micro perforations that extend all the way through the micro perforation disc thereby forming thousands of capillary like structures, separated-by the walls that are formed- when the micro perforations are made in the disc material. When assembled, the disc- body and the micro perforation disc are placed exterior to the fragrance-impregnated disc. The disc body and the micro perforation disc are slightly larger in diameter than the fragrance impregnated-disc and it is therefore possible to fix the two together at their correspondingly aligned circumferences. The actual technique selected for laminating the disc body and the micro perforated disc together as described, is left to those skilled in the art of manufacturing such plastic components and is not specifically a part of the present invention. The resulting sandwich allows the scent chemistry to stay intact within the inner part of the product with the only pathway for emission being the micro perforations. When the micro perforations are sealed with the plastic film, the scent chemistry remains essentially stabile until the plastic film is removed and the car air freshener is placed into service. It should be understood that all three components are alignable by their respective center holes. The mounting hole(s) are preferably created later after the components have been assembled, which allows them to be drilled without having to consider any alignment issues as would be the case if they were pre-drilled-in each of the components beforehand:

The car air freshener may be packaged as if it were a regular CD. Typically, CDs of various types are provided-in jewel cases that hold the CD in place through a clip that engages the center hole. The jewel case (not shown) is a conventional package and comes in standard dimensions for use in CD storage cases and the like. This can be done since the plastic film on the micro perforation disc acts to seal-the fragrance. The car air freshener would be supplied with the plastic film intact and-giving it sufficient shelf life to allow the user to deploy it when it is ultimately needed.

The car air freshener has an elastic strap (or loop) for hanging from a fixture in a motor vehicle, such as the mirror or a knob. The usage of the, elastic is conventional and expands over some parts of a fixture, such as the mirror, and allows the car air freshener to be hung securely. The ideal is to hang the car air freshener where it can be affected-by airflows. This will help to spread the scented fragrance that is being emitted from the product.

The unique approach taken in the packaging of this product results in several benefits. As mentioned above, the ability to conveniently store the car air freshener in a typical CD case, CD holder, CD rack, or the like, means that one always knows where to look for the product when it is needed. More than one stored car air freshener may be desired, especially if a person is heading out for a long road trip or if there are other conditions where comfort or preference dictate the use of the product on a frequent basis. The fact that one or more car air fresheners can be easily stored in units that are already available as accessory or OEM installations is a benefit of the present invention.

The offering of a car air freshener in a CD like configuration acts as a platform for cosmetic or marketing functions. For instance, the disc, body can easily be supplied with colors, patterns, or images that correspond to the likes of the consumer. Affinity marketing can be done whereby the car air freshener can be associated with the release of a specific music CD for instance. Other affinity approaches include the labeling of the product with sports teams, college teams, or any other favorite image of the user. This makes the car air freshener an exceptionally easy and convenient vehicle for use in promoting teams, or schools and can be use&as part of their fund-raising efforts.

The application of the present invention can be extended to uses other than promoting the emission of pleasing fragrances in confined areas. Animal related scents, such as those used by hunters who are pursuing deer or elk for instance, can be substituted instead, allowing the product to be used in the field for attracting the desired game (animals); Such uses may dictate that the outside appearance of the product be given a camouflage treatment so as to avoid the possibility of spooking the game if using a shiny, reflective surface which is normally found in the CD type discs. The advantages of the present invention transfer quite welt into this application for many of the same reasons. The product can be packaged to be stored easily, and several can be held in 15 place for ready use. Actual usage is a repeat of the method for deploying the car air freshener, except the product can be hung from a tree.

The foregoing attributes of the present invention are illustrated for the purposes of showing how it may be used: None of the teachings herein are intended to be limiting in 20 any way and reasonable variations and extensions of the use of the present concept are anticipated as comprising a part of the scope of the invention.

I claim:

1. A car air freshener, constructed to simulate the appearance of a compact disc (CD) and for use in providing a scented fragrance within the cabin of a vehicle, comprising:
A disc body;
A fragrance impregnated disc containing scent chemistry;
A micro perforated disc; and,
Where said disc body and said micro perforated disc enclose said fragrance impregnated disc there between, and where the further inclusion of a removable plastic film covering the exterior surface of said micro perforated disc the assembly substantially seals the scent chemistry and prevents the emission of a scented fragrance until such time as the plastic film is removed.

2. The car air freshener of claim Number 1, where the car air freshener includes a mounting hole and an elastic strap inserted there through for hanging the car air freshener.

3. The car air freshener of claim Number 1, where the fragrance impregnated disc is comprised of a felt saturated with the scent chemistry.

4. The car air freshener of claim Number 1, where the car air freshener is packaged in a jewel case.

5. A car air freshener, constructed to simulate the appearance of a compact disc (CD) and for use in providing a scented fragrance within the cabin of a vehicle, comprising:
A disc body;
A fragrance impregnated disc containing scent chemistry;
A micro perforated disc;
A jewel case; and,
Where said disc body and said micro perforated disc enclose said fragrance impregnated disc there between, and where the further inclusion of a removable plastic film covering the exterior surface of said micro perforated disc the assembly substantially seals the scent chemistry and prevents the emission of a scented fragrance until such time as the plastic film is removed, and where the assembly includes a mounting hole and a center hole, and where the assembly is storable in the jewel case until the car air freshener is needed for use.

6. The car air freshener of claim Number 5, where it further includes an elastic strap inserted into the mounting hole, for hanging the car air freshener in a desired location.

7. The car air freshener of claim Number 5, where the fragrance impregnated disc is comprised of a felt saturated with the scent chemistry.

8. An air freshener, constructed to approximate the size of a compact disc (CD) and for use in providing a scented fragrance, comprising:
A disc body;
A fragrance impregnated disc containing scent chemistry;
A micro perforated disc;
A jewel case; and,
Where said disc body and said micro perforated disc enclose said fragrance impregnated disc there between, and where the further inclusion of a removable plastic film covering the exterior surface of said micro perforated disc the assembly substantially seals the scent chemistry and prevents the emission of a scented fragrance until such time as the plastic film is removed, and where the assembly includes a mounting hole and where the assembly is storable in the jewel case until the air freshener is to be used.

9. The air freshener of claim Number 8, where the fragrance impregnated disc is comprised of a felt saturated with the scent chemistry.

10. The air freshener of claim Number 8, where the scent chemistry is comprised of scent that attracts game.

11. The car air freshener of claim Number 8, where the air freshener is camouflaged.
